(19) United States
(12) Patent Application Publication
(10) Pub. No.: US 2025/0349080 A1
(43) Pub. Date: Nov. 13, 2025

(54) METHOD AND APPARATUS FOR GENERATING THREE DIMENSIONS LIVER IMAGES

(71) Applicant: SAMSUNG LIFE PUBLIC WELFARE FOUNDATION, Seoul (KR)

(72) Inventors: Jin Soo RHU, Seoul (KR); Jae Won JOH, Seoul (KR); Gyu Seong CHOI, Seoul (KR); Jong Man KIM, Seoul (KR); Woo Kyoung JEONG, Seoul (KR); Jae Hun KIM, Seoul (KR); So Young LIM, Seoul (KR)

(21) Appl. No.: 19/223,684

(22) Filed: May 30, 2025

Related U.S. Application Data
(63) Continuation-in-part of application No. PCT/KR2023/019306, filed on Nov. 28, 2023.

(30) Foreign Application Priority Data
Dec. 1, 2022 (KR) 10-2022-0166097

(51) Int. Cl. G06T 19/00; G06V 10/25; G06V 10/764; G06V 10/82; G06V 20/64

(52) U.S. Cl. CPC G06T 19/00; G06V 10/25; G06V 10/764; G06V 10/82; G06V 20/64; G06T 2210/41; G06V 2201/031

(57) ABSTRACT

Provided is a method of reconstructing a three-dimensional (3D) liver image, which includes: receiving, by an image processing device, a two-dimensional (2D) liver image; inputting, by the image processing device, the 2D liver image into an image model; and reconstructing, by the image processing device, a 3D liver image corresponding to the 2D liver image using the image model, wherein a region of the 3D liver image is classified into a region of liver parenchyma, a region of a hepatic vein, a region of a portal vein, and a region of a background, when a mass is included in the region of the 3D liver image, the region of the 3D liver image is further classified to include a region of mass, and the region of the liver parenchyma is divided into at least one of a region occupied by a left portal vein, a region occupied by a right portal vein, a region occupied by a left hepatic vein, a region occupied by a middle hepatic vein, a region occupied by a right hepatic vein, a region occupied by an inferior hepatic vein, and a region occupied by vascular branches.

### CROSS-REFERENCE TO RELATED APPLICATION

This application claims the benefit under 35 USC 119 (a) of Korean Patent Application No. 10-2022-0166097 filed on Dec. 1, 2022, in the Korean Intellectual Property Office, and PCT application PCT/KR2023/019306 filed on Nov. 28, 2023, in the World Intellectual Property Organization, the entire disclosure of which is incorporated herein by reference for all purposes.

### BACKGROUND

### 1. Field

The technology described below relates to a method of reconstructing a three-dimensional liver image from a two-dimensional liver image.

### 2. Discussion of Related Art

The liver is a solid organ in the human body and has a highly complex vascular structure. When performing liver transplantation, it is important to anatomically and accurately understand the vascular structures of the liver. In order to accurately understand the vascular structures, computed tomography (CT) images or magnetic resonance imaging images are used. However, it is difficult to accurately identify the three-dimensional structure of blood vessels using these images alone.

### SUMMARY

Recently, methods for reconstructing three-dimensional (3D) liver images using two-dimensional (2D) liver images have been developed. However, conventional methods are not automated. Experts need to manually reconstruct 3D liver medical images using specific software. This takes much time and effort.

The technology described herein provides a method of reconstructing a 3D liver image from a 2D liver image using a model implemented through deep learning. In particular, the technology described herein provides a method of automatically classifying and displaying territories occupied by structures of each hepatic vein of the liver.

### Technical Solution

A method of reconstructing a three-dimensional (3D) liver image includes: receiving, by an image processing device, a two-dimensional (2D) liver image; inputting, by the image processing device, the two-dimensional liver image into an image model; and reconstructing, by the image processing device, a 3D liver image corresponding to the 2D liver image using the image model, wherein a region of the 3D liver image is classified into liver parenchyma, hepatic vein, portal vein, and background. When a mass is included in the 3D liver image, it is further classified to include a mass region image is further classified to include a region of mass, and the region of the liver parenchyma is divided into at least one of a region occupied by a left portal vein, a region occupied by a right portal vein, a region occupied by a left hepatic vein, a region occupied by a middle hepatic vein, a region occupied by a right hepatic vein, a region occupied by an inferior hepatic vein, and a region occupied by vascular branches.

The technology described herein provides a method of reconstructing a 3D liver image from a 2D liver image.

The technology described herein provides a method of automatically classifying liver parenchyma, hepatic veins, and hepatic arteries in 3D liver images.

The technology described herein provides a method of displaying liver parenchyma in a 3D liver image divided according to territories occupied by each vein.

### BRIEF DESCRIPTION OF THE DRAWINGS

FIG. 1 illustrates a process of generating a 3D liver image from a 2D liver image.

FIG. 2 illustrates a process of preparing training data.

FIG. 3 show 2D liver CT images used as training data.

FIG. 4 show 3D liver CT images used as training data.

FIG. 5 show 2D liver CT images used as training data.

FIG. 6 show 3D liver CT images used as training data.

FIG. 7 show 3D liver CT images used as training data.

FIG. 8 illustrates a configuration of an image processing device.

### DETAILED DESCRIPTION

Since the technology to be described below can have various changes and can have various embodiments, specific embodiments are illustrated in the drawings and described in detail. However, this is not intended to limit the technology described below to specific embodiments, and it should be understood to include all changes, equivalents, and alternatives falling within the spirit and scope of the technology described below.

The terms “first,” “second,” “A,” “B,” etc., may be used to describe various components, but the components are not limited by the terms, which are only used to distinguish one component from another. For example, without departing from the scope of the following description, a first component may be referred to as a second component, and similarly, a second component may also be referred to as a first component. The term “and/or” includes any and all combinations of one or more of the associated listed items.

As used herein, the singular forms should be understood to include the plural forms unless the context clearly indicates otherwise, and the terms “comprises,” “comprising,” “includes,” and/or “including,” when used herein, specify the presence of stated features, integers, steps, operations, elements, components and/or groups thereof, and do not preclude the presence or addition of one or more other features, integers, steps, operations, elements, components and/or groups thereof.

Before describing the drawings in detail, it should be clarified that the division of constituent parts in this specification is merely a division by main functions of each constituent part. That is, two or more constituent parts to be described below may be combined into one constituent part, or one constituent part may be divided into two or more constituent parts for each subdivided function. In addition, each of the constituent parts described below may additionally perform some or all of the functions of other constituent parts in addition to the main function of the constituent part itself, and it goes without saying that some of the main functions performed by each of the constituent parts may be performed exclusively by other components.

Also, in performing a method or an operation method, processes constituting the method may take place differently from the stated order unless clearly specified in the context. That is, each process may occur in the same order as described, may be performed substantially simultaneously, or may be performed in reverse order.

In the technology described below, a training model is a machine learning model, and a machine learning model may be various types of models. For example, a training model may be a deep learning model for image generation.

In the technology described below, medical images include computed tomography (CT) images, positron emission tomography (PET) images, magnetic resonance imaging (MRI) images, and ultrasound images.

In the technology described below, a liver CT image may be a CT image of the liver of a patient who has been administered a contrast agent. A liver CT image may be distinguished into an arterial phase and a portal phase depending on the circulation time in the body after the administration of the contrast agent.

In the technology described below, the liver may be divided into a hepatic artery, a portal vein, a hepatic vein, and a liver parenchyma.

In the technology described below, the portal vein may be divided into a right portal vein (RPV) and a left portal vein (LPV).

In the technology described below, the hepatic vein may be divided into a left hepatic vein (LHV), a middle hepatic vein (MHV), a right hepatic vein (RHV), an inferior vena cava, and inferior hepatic vein (IHV).

An overall process in which an image processing device 100 reconstructs a 3D liver image from 2D liver images will be described.

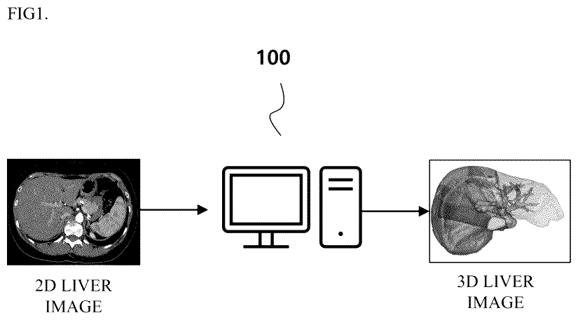

FIG. 1 illustrates a process of reconstructing a 3D liver image from a 2D liver image.

An image processing device 100 may receive a 2D liver image as input. The image processing device 100 may input the 2D liver image into an image model. The image processing device 100 may reconstruct a 3D liver image corresponding to the 2D liver image using the image model. The image processing device 100 may classify regions of the 3D liver image into a liver parenchyma, a hepatic vein, a portal vein, a mass, and a background using the image model. The image processing device 100 may output the 3D liver image in which each region is classified.

The 2D liver image may be a medical image of the liver. The medical image may be at least one of an MRI image and a CT image. For convenience of explanation, the following description will be provided based on an example in which 2D liver images are CT images.

The image model may be a model that receives 2D liver images as input and reconstructs 3D liver images. The image model may be a model implemented using artificial neural networks. The image model may be a model implemented with a U-net, which is a type of deep learning model. The U-net model may include an encoder, a decoder, and an output layer.

Input data of the image model may be a 2D liver image. The 2D liver images may be images captured from multiple views (axial, coronal, and sagittal views).

The output data of the image model may be a 3D liver image. The 3D liver image may be an image corresponding to the 2D liver images received as input data.

The image model may output the 3D liver image in which regions of the liver are classified.

The image model may output the 3D liver image that is classified into at least one of a liver parenchyma, a hepatic vein, a portal vein, a mass, and a background.

The image model may distinguish respective regions of the liver with different colors.

The image model may configure the output layer to output five output values. The five output values may be at least one of the liver parenchyma, the hepatic vein, the portal vein, the mass, and the background. The 3D liver image output by the image model may have a voxel resolution of 160 mm×160 mm×80 mm.

The image model may output the liver parenchyma with vein territories. The vein territories may include at least one of a territory occupied by the left hepatic vein, a territory occupied by the middle hepatic vein, a territory occupied by the right hepatic vein, a territory occupied by the inferior hepatic vein, and a territory occupied by vascular branches in the middle hepatic vein territory. Alternatively, the image model may output the liver parenchyma with a territory occupied by the left portal vein and a territory occupied by the right portal vein.

A process of training the image model may be performed using a training device. The training device may be a device capable of data processing such as a PC, a laptop, or a server.

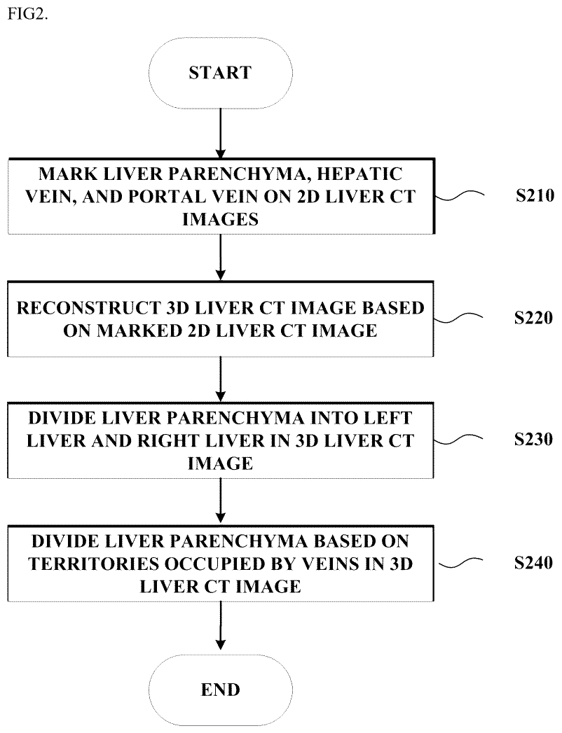

FIG. 2 illustrates a process by which a user builds training data for training an image model.

The user may acquire 2D liver CT images to be used as training data. 2D and 3D liver CT images may be images captured using contrast CT. 2D liver CT images may include images captured in the arterial phase and the portal phase.

The user may mark the hepatic artery on the CT images captured in the arterial phase among the 2D liver CT images. In addition, the user may mark the portal vein for reference.

The user may mark the liver parenchyma, the hepatic vein, and the portal vein on the CT images captured in the portal phase among the 2D liver CT images (S210).

The user may reconstruct the 2D liver CT images into a 3D liver CT image (S220).

Medical imaging programs may be used in this process. The medical imaging programs may include Mimics Medical by Materialise, MEDIP by Medical IP, Aview by Coreline Soft, and open-source programs such as 3D Slicer and InVesalius. The 3D liver CT image may be reconstructed using a semi-automated function of the medical imaging programs together with manual operations.

The user may reconstruct the 3D liver CT images using the liver parenchyma, the portal vein, and the hepatic vein marked on the 2D liver CT images. Alternatively, the user may segment the liver parenchyma, the portal vein, and the hepatic vein in the 3D liver CT image using the liver parenchyma, the portal veins, and the hepatic veins marked on the 2D liver CT images.

The user may divide the liver parenchyma into a left liver and a right liver in the reconstructed 3D liver CT image (S230). The user may use four reference points to distinguish between left and right liver.

The four reference points for distinguishing the left and right liver are as follows: 1) a groove between the middle hepatic vein and the right hepatic vein; 2) a groove between branches of the right portal vein and the left portal vein; 3) a central line of the inferior vena cava trunk; and 4) a boundary formed by branches of the left portal vein and the right portal vein in the center

The user may distinguish the liver parenchyma based on territories occupied by veins after dividing the liver parenchyma into the left liver and the right liver (S240).

The user may distinguish the liver parenchyma based on the vein territory. The user may divide the liver parenchyma in the order of 1) a territory occupied by the left hepatic vein, 2) a territory occupied by the middle hepatic vein, 3) a territory occupied by the right hepatic vein and the inferior hepatic vein, 4) a territory occupied by vascular branches V4, V5, and V8 in the middle hepatic vein territory, and 5) territories occupied by the right hepatic vein and the inferior hepatic vein in the right hepatic vein and inferior hepatic vein territories.

The 2D liver CT images and the 3D liver CT images in which the liver parenchyma is delineated may be used as training data.

The following describes embodiments of the constructed training data.

In the embodiment, the images used as training data include CT angiography images of 95 prospective liver donors of Korean nationality, which were captured for pre-examination purposes for living donor liver transplantation. The images used as training data have 2D images with a resolution of 512×512 pixels, and the Z-axis direction (the number of slices) varies for each patient. The CT images were labeled by identifying or classifying portal veins, hepatic veins, and liver parenchyma. In CT images, the liver parenchyma was subdivided and labeled based on the left lobe, the right lobe, and the major venous branches. More specifically, the CT images were labeled (classified) into a right superior hepatic vein territory, a right inferior hepatic vein territory, a V5 territory, a V8territory, a V4a territory, a V4b territory, and a left hepatic vein territory. The CT images were also separately labeled for the Spigelian lobe.

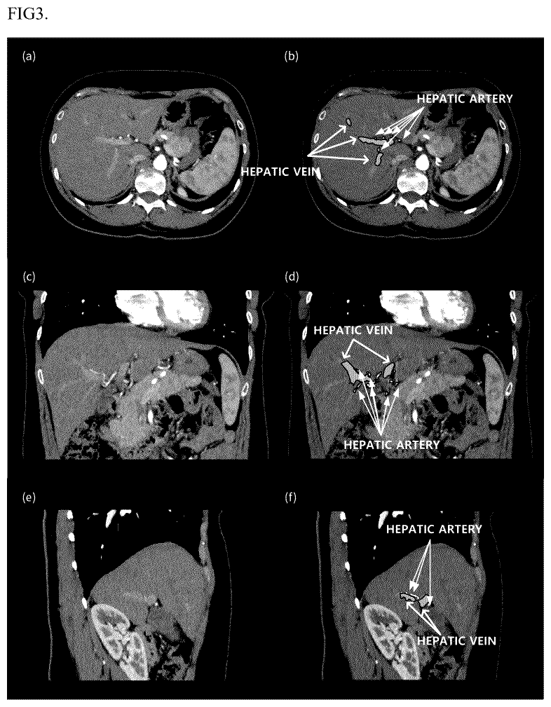

FIG. 3 show 2D liver CT images used as training data. FIG. 3 shows 2D liver CT images captured in the arterial phase.

FIGS. 3(a)(b) are CT images shown in the axial view. FIGS. 3(c)(d) are CT images shown in the coronal view. FIGS. 3(e)(f) are CT images shown in the sagittal view.

FIGS. 3(a)(c)(e) are original 2D liver CT images.

FIGS. 3(b)(d)(f) are 2D liver CT images in which hepatic veins and hepatic arteries are indicated. As shown in FIGS. 3(b), (d), and (f), the hepatic veins and hepatic arteries are distinguished and displayed in the 2D liver CT images. More specifically, the hepatic veins are displayed as areas with dotted interiors (an arrow shaped like “−>”), and the hepatic arteries are displayed as areas with white interiors (an arrow shaped like “ [symbol] ”). The 2D CT images with the hepatic veins and the hepatic arteries indicated are reconstructed into 3D CT images.

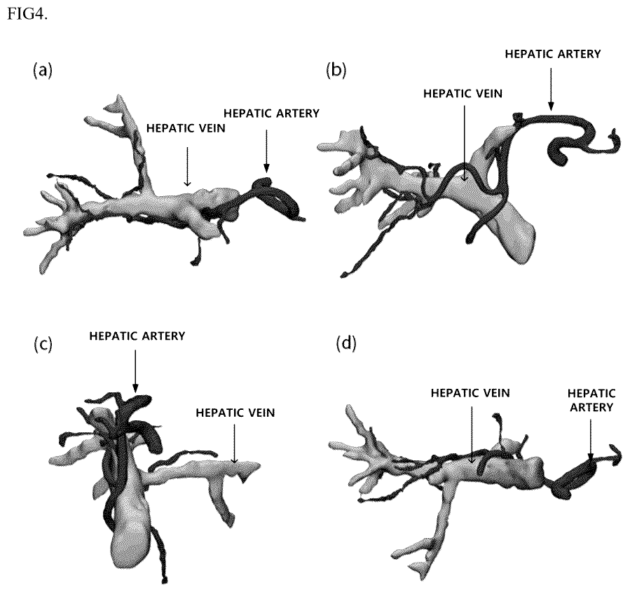

FIG. 4 show 3D liver CT images used as training data. FIG. 4 show 3D liver CT images corresponding to 2D liver CT images captured in the arterial phase. FIG. 4 show 3D liver CT images reconstructed from 2D CT images.

FIG. 4(a) is a 3D image viewed from above. FIG. 4(b) is a 3D image viewed from the front. FIG. 4(c) is an image viewed from the right side. FIG. 4(d) is a 3D image viewed from below.

Hepatic veins are displayed as gray areas (an arrow shaped like “−>”) and hepatic arteries are displayed as darker areas (an arrow shaped like “ [symbol] ”).

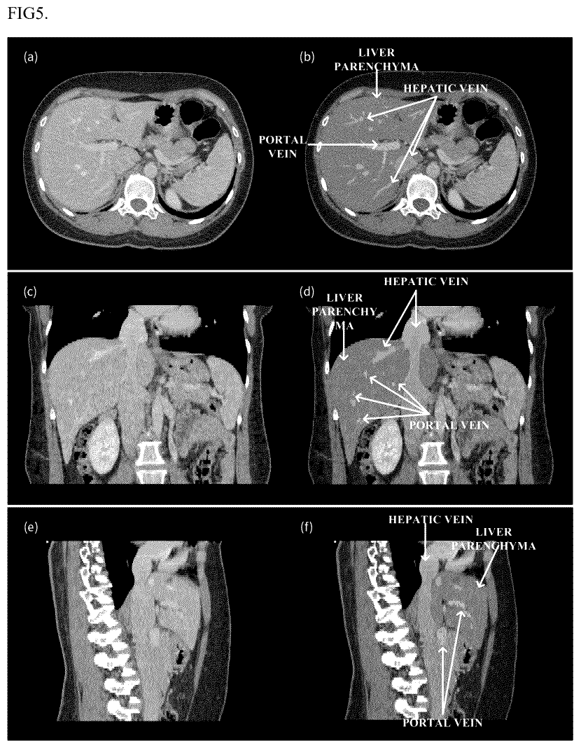

FIG. 5 show 2D liver CT images used as training data. FIG. 5 are CT images captured in the portal phase.

FIGS. 5(a)(b) are CT images shown in the axial view. FIGS. 5(c)(d) are CT images shown in the coronal view. FIGS. 5(e)(f) are CT images shown in the sagittal view.

FIGS. 5(b)(d)(f) are images of 2D liver CT images in which liver parenchyma, hepatic veins, and portal veins are indicated. The liver parenchyma, the hepatic veins, and the portal veins are distinguished in the liver CT images. The 2D CT images with the liver parenchyma, the hepatic veins, and the portal veins indicated are reconstructed into 3D CT images.

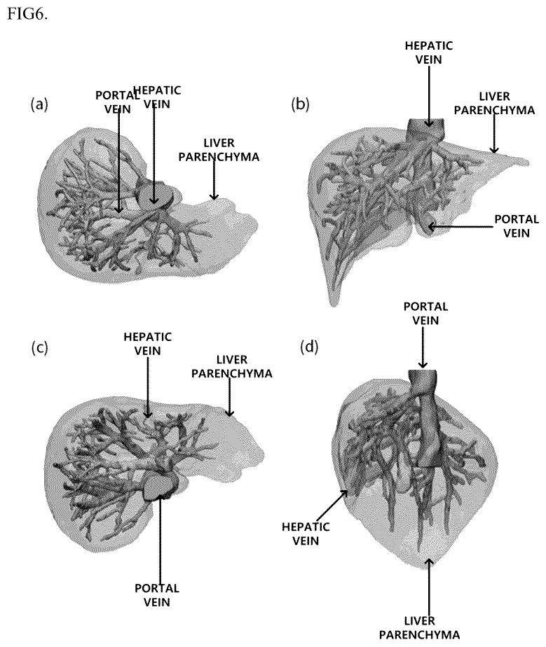

FIG. 6 shows 3D liver CT images used as training data. FIG. 6 show 3D liver CT images corresponding to 2D liver CT images captured in the arterial phase. That is, FIG. 6 show 3D liver CT images reconstructed from 2D CT images.

FIG. 6(a) is a 3D image viewed from above. FIG. 6(b) is a 3D image viewed from the front. FIG. 6(c) is an image viewed from the right side. FIG. 6(d) is a 3D image viewed from below.

In the 3D liver images, the liver parenchyma, the hepatic veins, and the portal veins are displayed with different shades. Masses are not separately displayed.

In 3D liver CT images, the liver parenchyma is delineated according to the vein territories.

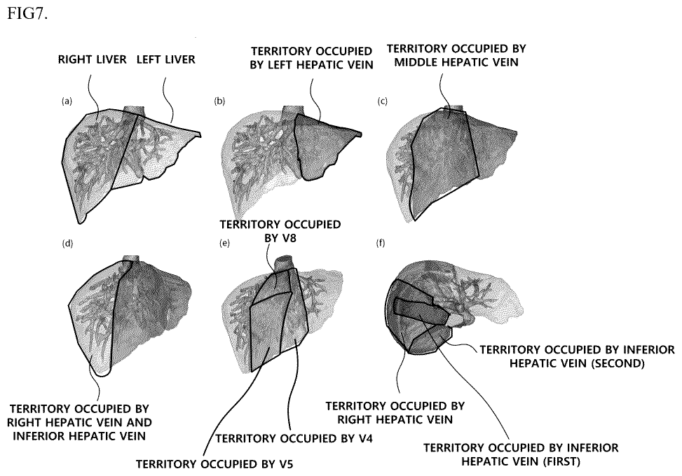

FIG. 7 show images in which the liver parenchyma is divided into the territory occupied by the right portal vein, the territory occupied by the left portal vein, the territory occupied by the left hepatic vein, the territory occupied by the middle hepatic vein, the territory occupied by the right hepatic vein, and the territory occupied by branch veins.

FIG. 7(a) is an embodiment in which the liver parenchyma is classified as the right liver or left liver.

The liver parenchyma is divided into the left and right liver and then divided based on the vein territories.

FIG. 7(b) is an embodiment in which the territory occupied by the left hepatic vein in the liver parenchyma is separately displayed.

FIG. 7(c) is an embodiment in which the territory occupied by the middle hepatic vein in the liver parenchyma is separately displayed.

FIG. 7(d) is an embodiment in which the territories occupied by the right hepatic vein and the inferior hepatic vein in the liver parenchyma are separately displayed.

FIG. 7(e) is an embodiment in which the territories occupied by branch veins V4, V5, and V8 in the territory occupied by the middle hepatic vein in the liver parenchyma are separately displayed. Areas belonging to the branch veins V4, V5, and V8 are displayed with different shades. V4 may be a branch vein coming from segment 4. V5 may be a branch vein coming from segment 5. V8 may be a branch vein coming from segment 8. V5 and V8 may combine to form the middle hepatic vein.

FIG. 7(f) is an embodiment in which the territories occupied by the right hepatic vein and the inferior hepatic vein are separately displayed. The territory occupied by the right hepatic vein and the territories occupied by the inferior hepatic vein (first and second territories) are displayed as separate areas.

A process in which the training device builds an image model using training data will be described below.

The training device preprocessed 3D liver CT images.

The training device reconstructed the 3D liver CT images to have a voxel resolution of 1.5 mm×1.5 mm×2.0 mm. From the reconstructed 3D liver CT images, only voxels having HU values in a range of −300 to 300 were extracted. The training device converted the HU values of extracted voxels to a range between 0 and 1.

The training device expanded the training data by flipping, rotating, and scaling the 3D liver CT images.

The training device expanded the training data by randomly dividing the 3D liver CT images with a size of 250×250×120 into images with a size of 160×160×80.

90% of the training data was used to train the image model. 10% of the training data was used as validation data.

The training device built an image model based on a U-net model, which is a type of artificial neural network model.

The training device constructed the encoder of the U-net model with a ResNet-based module. The training device constructed the decoder of the U-net model with a ConvNet-based module. The training device constructed the last layer of the U-net model to have five output values. The five output values may include at least one of background, liver parenchyma, hepatic vein, portal vein, and mass.

The training device trained the image model using Adam optimization and dice loss. The training device saved the weights of the deep learning model after 1000 training iterations.

The model trained as described above may receive 2D liver images as input and output 3D liver images. The trained model may also classify the output 3D liver image into liver parenchyma, hepatic vein, portal vein, hepatic artery, and mass. The trained model may also divide the liver parenchyma into the territory occupied by the left hepatic vein, the territory occupied by the middle hepatic vein, the territory occupied by the right hepatic vein, the territory occupied by the inferior hepatic vein, and the territory occupied by vascular branches in the middle hepatic vein.

The 3D liver image with divided regions of the liver provides medical staff performing liver transplantation with detailed anatomical knowledge of the liver. Medical staff performing liver transplantation may increase the success rate of liver transplantation through the obtained anatomical knowledge. Also, the automated methods may reduce significant time and effort.

Hereinafter, the configuration of the image processing device will be described.

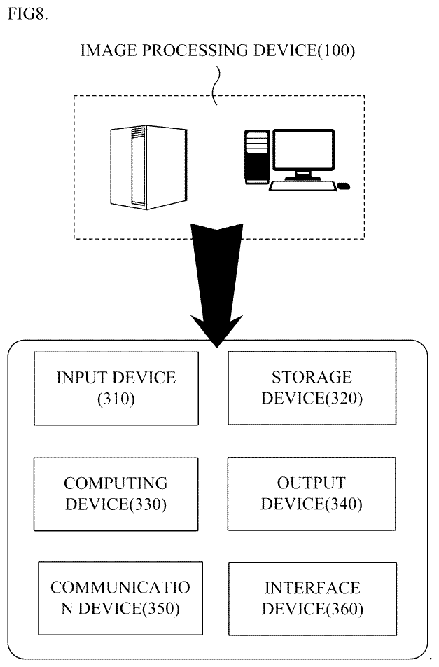

FIG. 8 illustrates the configuration of the image processing device.

The image processing device 100 corresponds to the image processing device shown in FIG. 1. Alternatively, the image processing device may include the training device described above.

The image processing device 100 may be physically implemented in various forms such as a personal computer (PC), a laptop, a smart device, a server, or a dedicated data processing chipset and the like. The image processing device 100 may include an input device 310, a storage device 320, and a computing device 330. The image processing device 100 may further include an output device 340, a communication device 350, and an interface device 360.

The input device 310 may include interface devices (e.g., a keyboard, a mouse, a touch screen, and the like) that receive certain commands or data. The input device 310 may also include a communication device 350 that receives and transmits certain information via wired or wireless networks. That is, the communication device 350 may be included in the input device. The input device 310 may include a component that receives information through separate storage devices (e.g., a USB, a CD, a hard disk, and the like). The input device 630 may also receive input data through a separate measurement device or through a separate DB. The input device 310 may receive building images as input. The input device 310 may receive 2D liver images as input.

The storage device 320 may store information from the input device 310. The storage device 320 may store an image model that reconstructs 3D liver images from 2D liver images. The storage device may store data generated during the processing by the image processing device 100. For this, memory may be included. The storage device 320 may store result values output by the image processing device 100. The storage device 320 may store training data used when training the image model.

The computing device 330 may reconstruct 3D liver images from 2D liver images using the image model. The computing device 330 may classify liver regions in the 3D liver image into at least one of a liver parenchyma, a hepatic vein, a portal vein, a mass, and a background.

The computing device 330 may classify liver parenchyma regions in the liver image into one of a region occupied by the left hepatic vein, a region occupied by the middle hepatic vein, a region occupied by the right hepatic vein, a region occupied by the inferior hepatic vein, and a region occupied by vascular branches in the middle hepatic vein.

The computing device 330 may train an image model using training data. The computing device 330 may build training data from received liver images. That is, the computing device 330 may perform the method shown in FIG. 2.

The output device 340 may be a device that outputs certain information. The output device may include a communication device 350 that receives and transmits information via wired or wireless networks. The output device 340 may output an interface required for data processes, input data, analysis results, and the like. The output device 340 may be physically implemented in various forms such as displays, document output devices, communication devices, and the like.

The communication device 350 may receive information from a separate device. Alternatively, the communication device 350 may transmit information generated by the image processing device 100 to a separate device.

The interface device 360 is a device that receives certain commands and data from the outside. The interface device 360 may receive 2D liver images from an input device physically connected thereto or an external storage device. The interface device 360 may receive training data as input. The interface device 360 may transmit 3D liver images to other objects. Meanwhile, the interface device 360 may include a component that receives data received by the communication device 350.

In addition, the above-described method of reconstructing a 3D liver image may be implemented as a program (or application) including an executable algorithm that can be executed on a computer. The program may be provided by being stored in a transitory or non-transitory computer readable medium.

A non-transitory readable medium is a medium that can store data semi-permanently and can be read by a device, rather than a medium that stores data for a short moment, such as a register, cache, or memory. Specifically, the above-described various applications or programs may be provided by being stored in a non-transitory readable medium, such as a compact disk (CD), a digital versatile disc (DVD), a hard disk, a Blu-ray disk, a Universal Serial Bus (USB), a memory card, a read-only memory (ROM), a programmable read only memory (PROM), an erasable PROM (EPROM), or an electrically erasable PROM (EEPROM), or a flash memory.

Transitory readable media include various RAMS, such as a static RAM (SRAM), a dynamic RAM (DRAM), a synchronous DRAM (SDRAM), a double data rate SDRAM (DDR SDRAM), an enhanced SDRAM (Enhanced) a Synclink DRAM (SLDRAM), and a direct rambus RAM (DRRAM).

The present embodiment and drawings attached thereto are only a part of the technical idea included in the above-described technology, and it is easy for a person skilled in the art to understand that modifications may be deduced within the scope of the technical idea included in the specification and drawings of the above-described technology and it will be appreciated that all specific embodiments are included within the scope of the above-described technology.

1. A method of reconstructing a three-dimensional (3D) liver image, the method comprising:
receiving, by an image processing device, a two-dimensional (2D) liver image;
inputting, by the image processing device, the 2D liver image into an image model; and
reconstructing, by the image processing device, a 3D liver image corresponding to the 2D liver image using the image model,
wherein a region of the 3D liver image is classified into a region of liver parenchyma, a region of a hepatic vein, a region of portal vein, and a region of background,
when a mass is included in the region of the 3D liver image, the region of the 3D liver image is further classified to include a region of mass, and
the region of the liver parenchyma is classified into a left region and a right region based on the portal vein and is classified into a region of a left hepatic vein, a region of a middle hepatic vein, and a region of a right hepatic vein divided by the hepatic vein.

2. The method of claim 1, wherein the region of the liver parenchyma further includes a region in which branch veins of the left hepatic vein are included, a region in which branch veins of the middle hepatic vein are included, and a region in which branch veins of the right hepatic vein are included.

3. The method of claim 1, wherein the image model is a model trained using 2D liver images and 3D liver images as training data, and
a region of liver parenchyma in the 3D liver image used as the training data is divided into at least one of a left region, a right region, a region of the left hepatic vein, a region of the middle hepatic vein, a region of the right hepatic vein, a region of vascular branches of the left hepatic vein, a region of vascular branches of the middle hepatic vein, and a region of vascular branches of the right hepatic vein.

4. The method of claim 1, wherein criteria for distinguishing the left region and the right region are a groove between the middle hepatic vein and the right hepatic vein, a groove between branches of a right portal vein and a left portal vein, a central line of an inferior vena cava, and a boundary formed centrally by the branches of the left portal vein and the right portal vein at a center.

5. The method of claim 1, wherein the image model is a model built using an artificial neural network.

6. The method of claim 5, wherein the model built using the artificial neural network is a model built based on a U-net model,
the U-net model includes an encoder, a decoder, and an output layer, and
the output layer classifies and outputs the region in the 3D liver image into at least one of a region of liver parenchyma, a region of a hepatic vein, a region of a portal vein, a region of a hepatic artery, and a region of background.

7. An apparatus for reconstructing a three-dimensional (3D) liver image, the apparatus comprising:
an input device configured to receive a two-dimensional (2D) liver image;
a computing device configured to reconstruct a 3D liver image corresponding to the 2D liver image using an image model; and
a storage device configured to store the image model,
wherein a region of the 3D liver image is classified into a region of liver parenchyma, a region of a hepatic vein, a region of a portal vein, and a region of background,
when a mass is included in the region of the 3D liver image, the region of the 3D liver image is further classified to include a region of mass, and
the region of the liver parenchyma is classified into a left region and a right region based on the portal vein and is classified into a region of a left hepatic vein, a region of a middle hepatic vein, and a region of a right hepatic vein divided by the hepatic vein.

8. A recording medium on which a program that causes a computer to perform the method of reconstructing a 3D liver image described in claim 1 is recorded.
